(19) United States
(12) Patent Application Publication
(10) Pub. No.: US 2013/0184566 A1
(43) Pub. Date: Jul. 18, 2013

(54) Elastic Antenna System for a Magnetic Resonance Imaging System

(71) Applicants: Ludwig Kreischer, Dormitz (DE); Volker Matschl, Bamberg (DE)

(72) Inventors: Ludwig Kreischer, Dormitz (DE); Volker Matschl, Bamberg (DE)

(21) Appl. No.: 13/743,164

(22) Filed: Jan. 16, 2013

(30) Foreign Application Priority Data
Jan. 17, 2012 (DE) DE 102012200599.4

(51) Int. Cl. G01R 33/34; A61B 5/055; G01R 33/48

(52) U.S. Cl. CPC G01R 33/34092; G01R 33/48; A61B 5/055
USPC 600/422; 324/322; 324/309

(57) ABSTRACT

An antenna system for a magnetic resonance imaging system includes a plurality of antenna elements. The antenna elements are arranged in, at, or on support elements. The support elements are constructed so as to be non-expandable and have a constant surface dimension. Adjacent support elements are connected to an expandable connecting element. The dimensions of the connecting element may be changed by the expansion.

This application claims the benefit of DE 10 2012 200 599.4, filed Jan. 17, 2012, which is hereby incorporated by reference.

### BACKGROUND

The present embodiments relate to an antenna system for a magnetic resonance imaging system.

Focuses of development for modern magnetic resonance imaging systems are the improvement in a signal-to-noise ratio of a magnetic resonance signal and, for example, possibilities for parallel detection of a magnetic resonance signal. For example, the strength of a main or basic magnetic field, which is used for homogeneous basic orientation of magnetic dipoles of an object to be examined, is brought to a strength of several tesla for this purpose. A further possibility of improving the signal-to-noise ratio of magnetic resonance signals and the imaging quality of a magnetic resonance image lies in an advantageous design and arrangement of the transmitting or receiving antenna systems (e.g., transmitting or receiving coils) of the magnetic resonance imaging system for initiating or receiving a magnetic resonance signal. By optimizing the position of the transmitting or receiving coils, the filling factor of this antenna system may be improved, and this indicates the ratio of the volume of an object to be examined to the total volume that is detected by the antenna system. To improve the filling factor, which may be assumed to be proportional to the square of the signal-to-noise ratio, it is advantageous to arrange the transmitting and receiving coils (i.e., local coils) in the immediate vicinity of the object to be examined so as to follow the surface of the object to be examined.

Limits are set on the approximation of the surface, however, in the case of sections of the surface of the object to be examined that have a complex shape. For magnetic resonance imaging of the human body, different local coils are known, for example, which assume roughly the form of a section of the body (e.g., the form of a knee or a hand). To avoid a large number of different local coils having to be kept in stock, special elastic antenna elements, for example, are possible for magnetic resonance imaging of these sections of the body. These are connected to a stocking-like or glove-like support that closely and expandably surrounds the object to be examined. The antenna elements follow the change in shape of the support, so reliable operation of the shape-changed antenna elements uses extensive compensation measures. For example, the change in the shape of the transmitting or receiving coils changes the capacitance or even inductance, so compensation of this change to give a reliable setting of the resonance frequency is to be provided.

### SUMMARY AND DESCRIPTION

The present embodiments may obviate one or more of the drawbacks or limitations in the related art. For example, an improved antenna system, magnetic resonance imaging system, and method for the acquisition of magnetic resonance signals are provided.

In one embodiment, an antenna system for a magnetic resonance imaging system includes a plurality of antenna elements. The antenna elements are connected to the support elements allocated to the individual antenna elements, which are not expandable, so the support elements have a substantially constant surface dimension (e.g., a surface measure) that the support elements retain, for example, even during a flexible change in shape. “Substantially constant” in this context provides that a change in the surface dimension is limited to thermal changes, age-related changes in material or the like. Adjacent antenna element support elements are also connected by an expandable connecting element. The dimensions of the connecting element may be changed by way of the expansion. For example, the spacing of the support elements from each other may also be changed by expansion of the connecting element. The connecting elements may each be constructed as separate parts between the individual support elements. In one embodiment, a plurality of support elements may be connected by one connecting element, or integrated support elements and connecting elements may be implemented.

The antenna system may be constructed so as to directly follow the surface shape of an object to be examined. For example, a knee or hand may be closely surrounded by the antenna system, and transmitting and receiving properties may be optimally adjusted to the object to be examined. For example, the signal-to-noise ratio may be improved. An advantageous filling factor may be achieved. The surface dimension of the support element and a “loop size” of the antenna element (e.g., the surface enclosed by a conductor loop of the antenna element, which forms the effective antenna surface) do not change, however, so extensive tuning measures that may result from a change in the dimensions of one of the antenna elements are omitted. For example, compensation of inductance or capacitance changes in the antenna elements is not provided or is easy to carry out. The operating expenditure of the antenna system is therefore particularly low.

The antenna system may, for example, involve both transmitting coils and receiving coils. The combined design as a transmitting and receiving coil system may also be provided. For example, one or more of the antenna elements may be used both for transmitting and for receiving in the antenna system.

A magnetic resonance imaging system is also provided. The magnetic resonance imaging system includes, in addition to the conventional components such as, for example, a basic field magnet, a gradient system, and optionally a permanently installed whole-body antenna, an above-described antenna system.

A method for the acquisition of magnetic resonance signals is also provided. High frequency (HF) signals are emitted, and magnetic resonance signals of an object to be examined are received. For transmitting the HF signals and/or for receiving the magnetic resonance signals, an antenna system that, as described above, includes a plurality of antenna elements is used. The antenna elements are connected to separate support elements that have a substantially constant surface dimension. An expandable connecting element is arranged between adjacent antenna elements. The dimensions of the connecting element may be changed by way of the expansion (e.g., changed from an initial or rest position to apply the antenna system to an object to be examined, a patient or test person).

The initial position or rest position corresponds to the configuration of the antenna system before mounting on an object to be examined (e.g., in a relaxed state of the expandable connecting elements).

Embodiments and developments result from the following description. One category of the description may also be developed analogously to another category of the description.

The support element is constructed so as to be flexibly formable (e.g., pliable). The surface dimension of the support element is not changed by the bending even if the surface shape of the support element may be changed. The adjustment options to different objects to be examined may therefore be drastically increased. In this case, the antenna element may be constructed so as to be flexible, so the antenna element follows the shaping of the support element. The loop size, for example, remains substantially constant even with the flexible change in shape of the support element. “Substantially constant” may be that the length of a conductor section that forms a loop or a winding of the antenna element remains unchanged except for thermal effects or changes in a similar order of magnitude. The compensation expenditure described above does not change as a result.

The connecting element may include an expandable film or is formed by an expandable film. The connecting element may be implemented so as to be flat, expandable and pliable by the film. Alternatively, an expandable fabric may also be used.

In a development, an at least two-dimensionally cohesive network of antenna elements is formed with the aid of a plurality of the connecting elements. An object to be examined may therefore be surrounded with the aid of the antenna system (e.g., in the form of an antenna array) over a large area so as to be at least partially closely surrounded.

The flexibility of the antenna system is determined by the ratio of the area of expandable regions to the area of regions with a constant surface dimension. The last-mentioned area is substantially determined by the area of the support elements or by the number of antenna elements. For example, the size of the antenna elements is determined following considerations, described in more detail below, relating to achieving an optimum signal-to-noise ratio.

In one embodiment, the number of support elements may be 8 to 15 for knee coils, 8 to 16 for shoulder coils, 8 to 36 for leg coils (e.g., PAA coil), 8 for ankle coils, 18 for arm coils, 12 for wrist coils, 18 for body coils, and 32 for spine coils. These allow an excellent signal-to-noise ratio.

To provide optimum flexibility of the antenna system, the expandability of the connecting elements may reach up to 50% of the size of the connected antenna element in the direction of expansion of the connecting element.

This network or array may, for example, include a plurality of similarly constructed support elements and/or similarly constructed antenna elements.

The support or antenna elements are, for example, arranged according to a rule (e.g., the support or antenna elements may be connected to connecting elements that have a similar construction to each other). Support elements and connecting elements may be regularly arranged in a row or in a matrix-like structure (e.g., at a certain grid spacing). This regularity relates, for example, to the arrangement of the antenna elements before mounting of the antenna system in or on an object to be examined (e.g., in the initial position). The antenna system may include a plurality of groups of different support elements or antenna elements that are each arranged according to a rule.

The antenna system may include substantially flat support elements, so the support elements may be placed directly adjacent to the object to be examined, for example, and enable a slight spacing of the antenna elements from the surface of the object to be examined. An antenna system that does not substantially protrude from the object to be examined may therefore be achieved, so positioning of the object to be examined in a magnetic resonance imaging system is not limited by a bulky form of the antenna system.

“Substantially flat” may be interpreted in this connection in that the extent of the support element in an antenna plane is at least twice as large as in a spatial direction orthogonal thereto. During operation of the antenna system, the antenna plane may be oriented parallel to the surface of the object to be examined. Substantially flat support elements may also be constructed so as to be pliable.

The support elements or antenna elements may also be regarded as being connected in an area, for example, so the antenna system has the basic form of a flat rectangle. For example, the antenna system may also be sheet-like in design, outstanding adjustment to the surface shape of the object to be examined being provided owing to the expandability of the connecting elements, for example.

In a development, the antenna system may include support elements that substantially follow the surface shape of a section of an object to be enclosed by the antenna system (e.g., the object to be examined). For example, the support elements may be constructed such that in certain sections, the support elements follow the surface shape of the object to be examined even in an initial position of the antenna system. The antenna system may thus be arranged so as to follow the surface of the object to be examined even better.

In the initial position, the antenna system may include curved support elements (e.g., curved in certain sections), which, for example, in certain sections, reproduce the shape of an object to be examined (e.g., a knee, heel or wrist) or follow this shape in certain sections. Even such curved support elements may be constructed so as to be pliable (e.g., may deviate from a curved starting form during operation of the antenna system).

With the aid of the curved support elements, the surface shape of part of an object to be examined or a group of similarly constructed objects to be examined (e.g., of hands, feet) may be reproduced, so the hold of the antenna system on the object to be examined, the adjustment options and, resulting therefrom, the signal-to-noise ratio, may again be further improved.

To assist adaptation to the shape of an object to be examined, the antenna system may include form fixing elements (e.g., changeable tensile and/or pressure elements). For example, this may be a belt with a hook and loop fastener that enables an arrangement of the antenna system that encloses the object to be examined. The expansion of the connecting elements during operation of the antenna system, for example, may therefore be adjustable, or fixing of the antenna system is enabled.

The tensile or pressure elements may be constructed as latches that define a series of preferred positions. The preferred positions may relate, for example, to the spacing of adjacent antenna elements.

In a development, the antenna elements and/or the antenna system are/is constructed for cableless or wireless operation (e.g., for wirelessly receiving information and/or power from a magnetic resonance imaging system), or also for wirelessly transmitting information to a magnetic resonance imaging system. In other words, the antenna system includes antenna wiring, so the antenna elements may be operated cablelessly. Therefore, no connecting cables are to be led across expandable sections of the antenna system. Therefore, connecting cables with an electrical length that then has to be compensated again during operation of the antenna system by way of corresponding expenditure may not be used.

In the case of wireless transmission, a plurality of the antenna elements may, for example, be constructed so as to be inductively coupled for receiving power to a further transmitting antenna arrangement of the magnetic resonance imaging system (e.g., the whole-body antenna (e.g., body coil) permanently installed in the tomograph). The antenna elements may receive an HF transmitting signal from the transmitting antenna arrangement of the magnetic resonance imaging system and radiate an object to be examined. Thus, the transmitting field is strengthened or modified, for example. Each of the antenna elements may include at least one tuning element for this purpose (e.g., a tunable capacitor).

In the case of wireless transmission, the antenna elements may also be constructed so as to be passively detunable in resonance frequency, so corresponding connecting cables may again be omitted. “Passively detuned” in this case provides that the power for controlling pin diodes that may be used for tuning or detuning the resonance frequency of individual antenna elements is taken from an HF transmitting field of a transmitting antenna arrangement of the magnetic resonance imaging system.

For wireless transmission in the antenna system, local preamplifiers may be allocated for the antenna elements, respectively, and the antenna system may also locally includes, for example, in or on the local coil, at least one analogue-to-digital converter, a modulator and a transmitter. These components are constructed as a whole for wireless transmission of information derived from a magnetic resonance signal.

The antenna system or the antenna elements may include a transmitting controller that is constructed to wirelessly receive information, so a transmitting coil system that may be operated with little expenditure is controlled on the basis of the received information.

The antenna system may, for example, include antenna elements that, in the initial position, have a spacing (e.g., a “gap”) from an adjacent antenna element. In connection with expandable connecting elements, safe decoupling of the individual antenna elements may thus be achieved during operation of the magnetic resonance imaging system, so, for example, with “under-sampled” magnetic resonance images, an improvement in the image quality may be achieved. For example, an optimum signal-to-noise ratio is provided in this connection. Minimum decoupling is provided due to this “gap” arrangement. Signal generation or evaluation for the coils may be separated better, so, for example, received signals may be easily allocated to individual coils. Adjacent antenna elements of the antenna system may have a minimum spacing that does not exceed approximately 20% of a coil diameter of the antenna element to provide the described type of decoupling.

However, an overlapping arrangement of the antenna elements may also be provided. For example, defined overlapping positions may be determined, for example, by a latching device that enables a predetermined residual coupling or decoupling of the antenna elements, with a flexible adjustment to the surface shape of an object to be examined still being provided. The overlapping positions may vary for this purpose, for example, such that up to 20% of the area enclosed by the antenna element overlaps. The overlapping positions may be adjustable in a grid in steps between 5 mm (e.g., for a wrist coil) and 10 mm (e.g., for a body coil) with, for example, the aid of the latching device. The latching device may be implemented, for example, in, on or through connecting elements that, in one spatial direction, has a tensile limit, for example, due to a sequence of latching noses.

The antenna system may be constructed such that the antenna system may be connected to a patient to be examined or test person or may be arranged before the patient or test person lowers himself or herself onto a couch of the magnetic resonance imaging system. Effective operation of the magnetic resonance imaging system is the consequence since, for example, while images are being made of a patient or test person using the magnetic resonance imaging system, one or more other patient(s) or test person(s) may already be fitted with appropriate antenna systems.

### BRIEF DESCRIPTION OF THE DRAWINGS

Identical components are provided with identical reference numerals in the various figures.

FIG. 1 shows a schematic diagram of one embodiment of a magnetic resonance imaging system;

FIG. 2 shows a schematic view of one embodiment of an antenna system;

FIG. 3 shows a plan view of one embodiment of an antenna system;

FIG. 4 shows a cross-section view of one embodiment of the antenna system of FIG. 3;

FIG. 5 shows a development of the exemplary embodiment of FIGS. 3 and 4;

FIG. 6 shows a plan view of one embodiment of an antenna system according to the overlap design;

FIG. 7 shows a cross-section view of one embodiment of the antenna system of FIG. 6;

FIG. 8 shows a further exemplary embodiment for an antenna system;

FIG. 9 shows one embodiment of an antenna system with curved support surfaces;

FIG. 10 shows one embodiment of an antenna system constructed for wireless operation; and

FIG. 11 shows a development of the exemplary embodiment of FIG. 10 in detail.

### DETAILED DESCRIPTION OF THE DRAWINGS

The diagrams in the figures, in particular the antenna system with the connections of the support elements by way of the expandable connecting elements, are only schematic and not to scale.

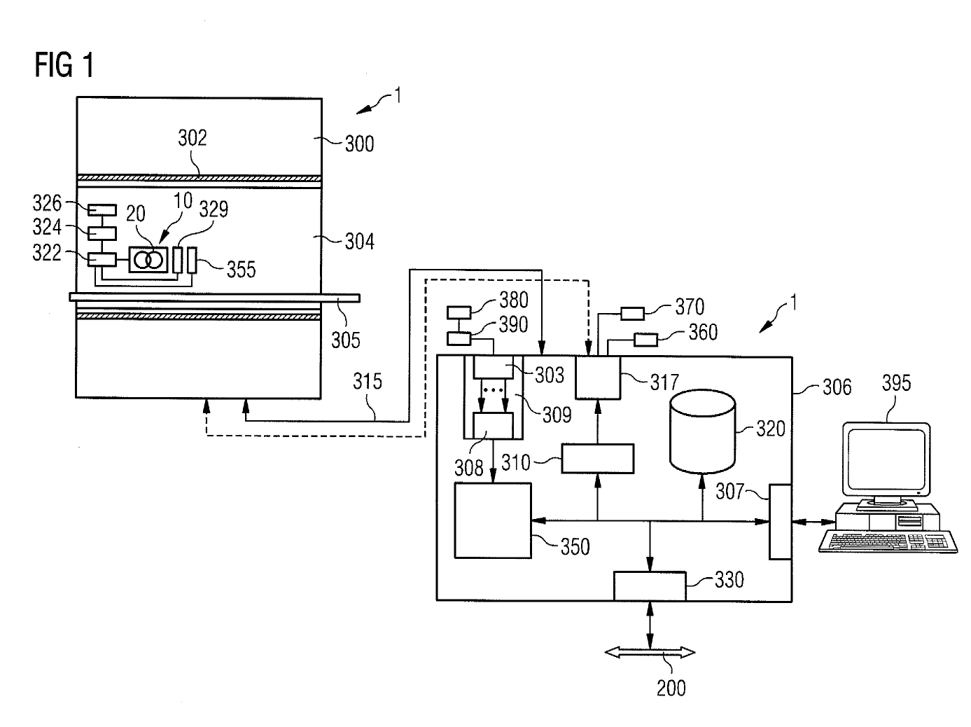

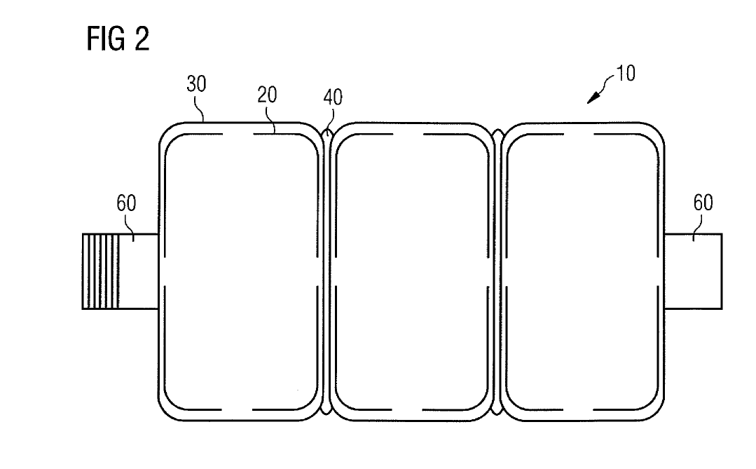

FIG. 1 shows one embodiment of a magnetic resonance system 1 including an antenna system 10 described in more detail with the aid of FIGS. 2 to 11. The magnetic resonance system I includes a commercially available tomograph 300 (e.g., a scanner 300), in which a patient (not shown) is positioned on a couch 305 in a cylindrical measuring space 304. Inside the tomograph 300 is a permanently installed whole-body antenna arrangement 302 that is constructed in the exemplary embodiment as a birdcage antenna for emitting magnetic resonance excitation signals or optionally also for receiving magnetic resonance signals.

In the exemplary embodiment, an antenna system 10 constructed as a local coil 10 includes a number of antenna elements 20. The local coil 10 is wirelessly connected to a transmission signal receiving module 303 of the magnetic resonance system 1. As FIG. 1 also shows, the local coil 10 is arranged in the measuring space 304 of the tomograph 300 of the magnetic resonance system 1, whereas the transmission signal receiving module 303 is implemented as part of a raw data acquisition interface 309 in an operation controller 306 of the magnetic resonance system 1.

Alternatively or in combination, the antenna system 10 may be connected to an operation controller 306 via a cable-based communication channel, as is also shown in FIG. 1 by the communication channel indicated by broken lines.

An MR signal processing device 308 is also part of the operation controller 306 or raw data acquisition interface 309, for example. The system may be scaled as desired (e.g., with appropriate configuration of the antenna system 10, any desired number of physical inputs of the MR signal processing device 308 may be served).

The tomograph 300 is also controlled by the operation controller 306. A terminal 395 (or an operator control board) is connected to the operation controller 306 by a terminal interface 307. An operator may operate the operation controller 306, and therewith the tomograph 300, via the terminal. The operation controller 306 is connected by a tomograph control interface 317 to the tomograph 300 to appropriately control the different components of the tomograph 300, such as the basic field magnet, the gradient system, the permanently installed high frequency transmitting system with the whole-body antenna arrangement 302, the couch 305, etc. This is symbolized by the supply line 315. Suitable control commands are output to the tomograph 300 via the tomograph control interface 317 via a sequence control unit 310 on the basis of scan protocols, so the desired pulse sequences (e.g., the high frequency pulse and the gradient pulse for the gradient coils (not shown) for generating the desired magnetic field gradient) are emitted.

The operation controller 306 also includes a memory 320, in which generated image data, for example, may be stored, and measuring protocols may be stored.

A further interface 330 is used for connecting to a communications network 200 that, for example, is connected to an image information system (e.g., a picture archiving and communication system (PACS)) or connecting options for external data storage.

The raw data is acquired (e.g., the received MR receiving signals read out) via the raw data acquisition interface 309, which includes, for example, the transmission signal-receiving module 303. The received signals are processed further in the MR signal processing device 308 and supplied to an image reconstruction unit 350 that conventionally generates the desired magnetic resonance image data therefrom. This may be stored, for example, in the memory 320, at least partially output on the terminal 395, or be transmitted via the communications network 200 to other components such as diagnostic stations or mass storage devices.

Both the operation controller 306 and the terminal 395 may be an integral component of the tomograph 300. The entire magnetic resonance system 1 also has all further conventional components or features of such a system, but these are not shown in FIG. 1 for the sake of improved clarity.

Since the local coil 10 in the exemplary embodiment is designed to communicate wirelessly with the operation controller 6, an instruction transmitting device 360 is also connected to the tomograph control interface 317, for example, and this wirelessly transmits instructions or control signals to the local coil arrangement 11.

A first power transmitting antenna 370 is also connected to the tomograph control interface 317, and this wirelessly sends power to a power receiving antenna 355 of the local coil 10 to supply the power receiving antenna 355 with power. The received power may be passed on, for example, to the local coil controller 322.

The local coil 10 with the antenna elements 20 is connected to an instruction receiving device 329 that receives the wirelessly transmitted instructions. The instructions are forwarded, for example, to the local coil controller 322. The local coil controller 322 supplies the antenna elements 20 with power and controls the antenna elements 20. MR receiving signals received from the local coil 10 are passed from the local coil controller 322 in prepared form (e.g., in digitized form) as MR transmission signals to a local coil transmitting device 324, from which the MR receiving signals are sent via a local coil transmitting antenna 326 to a receiving antenna 380 of the magnetic resonance system 1. The MR transmission signals received by the receiving antenna 380 are evaluated by a receiver 390 and supplied to the transmission signal receiving module 303.

The antenna system 10 constructed in the exemplary embodiment of FIG. 1 as a receiving antenna system is described in more detail below. The antenna system 10 described below may also be a transmitting antenna system 10 or an antenna system 10 with a combination of the functionalities. For this purpose, the local coils 10, as will also be shown later with the aid of exemplary embodiments, are provided with switching components to switch the local coils 10 from a wireless receiving mode into a wireless transmitting mode.

According to one embodiment, the antenna system 10 illustrated in more detail in FIG. 2 is constructed as a local coil 10 with an array including a plurality of similar antenna elements 20.

The local coil 10 also includes a plurality of similar support elements 30 (e.g., flat support boards) that have a substantially rectangular shape (e.g., with rounded corners) and, for example, a thickness perpendicular to a flat side of the support board between 5 mm and 20 mm. Arranged on each of the support boards is a respective antenna element 20 that is constructed with a conductor loop with capacitive elements (symbolized by the interruptions in the conductor loop on each side) and with wiring components for tapping a received magnetic resonance signal and for tuning or detuning the natural frequency with respect to a magnetic resonance frequency used. The capacitive elements and the wiring components are not shown for reasons of clarity.

The conductor loop of the antenna element 20 is arranged on or under a flat side of the support element 30 and follows the substantially rectangular shape of the support element 30 only insignificantly indented with respect to a limiting edge of the support element 30.

The conductor loop of the antenna element 20 therefore roughly assumes the dimensions of the support element 30. This coincidence of the dimensions or the only marginal differences in the external contours of the flat side of the support element 30 and the conductor loop bring about optimum flexibility and adjustability of the local coil to an object to be examined, even if the support elements 30, as in this case, are rigidly constructed as support boards. The adjustment to the surface shape of an object to be examined may improve the signal-to-noise ratio of a magnetic resonance signal.

In the exemplary embodiment, the conductor loop of the antenna element 20 forms a laminate with the respective support board that may enable a protected arrangement of the antenna element 20.

In one embodiment, the support elements 30 may be constructed as flat synthetic resin elements (e.g., in rigid board form or as a relatively thin film), into which, for of example, the antenna element 20 is molded. In each case, the conductor loop of the antenna element 20 has a constant “loop size (e.g., the area enclosed by the conductor loop is substantially constant).

The support boards may be made, for example, from Kapton or similarly flexible (conductor) board material or also from thin FR4 material (e.g., flame retardant, category 4). In one embodiment, the material may have a thickness up to 0.5 mm, so a durable connection to the antenna element is created to provide consistent signal quality.

In an alternative embodiment, the support elements 30 are constructed as a non-expandable fabric structure. The conductor loops of the antenna elements 20 may be “woven,” for example, into the fabric structure (e.g., the conductor loops penetrate the fabric several times over the course of the antenna element). Simple production is therefore enabled, with the respective antenna element 20 also being arranged so as to be protected.

Stretch materials such as, for example, textile fabrics that are flexible to a limited extent and have elastane components (e.g., elastane yarns) may be used as materials for the fabric structure.

As shown by FIG. 2, the support elements 30, and therefore the antenna elements 20, are connected to each other in a chain or chain-like arrangement by expandable connecting elements 40 (e.g., connecting films).

Expandability of the connecting films may be achieved in that the connecting film falls below the thickness of the support board.

The support boards and the connecting elements 40 may be arranged such that the antenna system 10 forms an approximately smooth, cohesive surface that faces the object to be examined.

The connecting film may be made, for example, of rubber, latex or films with similar flexibility to achieve the desired elasticity and that reliably return to the initial position.

In a further embodiment, the connecting elements 40 may also be formed by fabric materials that are flexible and expandable and also exhibit the advantages of the connecting film. Stretch materials (e.g., textile fabrics that are expandable to a limited extent and have elastane components (elastane yarns) and/or Dorlastan) may be used as the fabric materials.

In the exemplary embodiment of FIG. 2, a strip that is expandable with respect to overall length or an expandable chain is produced with the aid of the expandable connecting elements 40 with a plurality of antenna elements 20 that are each secured to a rigid support element 30. The expandable connecting film extends from one of the support elements 30 to the respective adjacent support element 30 or the next support element 30 in the chain. One of the side faces respectively of the support elements 30, substantially along the entire limiting edge of a side of the rectangle, forms a connecting face with the connecting element 40.

The chain may, for example, include at least four (e.g., at least eight) antenna elements 20 beyond the schematic diagram in FIG. 2.

This strip may be placed, for example, around a leg, an arm, a shoulder or a similarly complex object to be examined, enabling flexible adjustment to the anatomy of the object to be examined.

As shown in FIG. 2, the strip includes, at end members of the chain of support elements 30 and connecting elements 40, respectively, extension strips that are provided with hook and loop fastening elements, so the object to be examined may be closely and securely surrounded. The extension strips therefore serve as form fixing elements 60. An adjustment of the shape of the antenna system 10 to the shape of an object to be examined when applying and then fixing the antenna system 10 to the object to be examined occurs by way of the form fixing elements 60.

In combination with the expandability of the connecting film, a tensile stress that reliably prevents slippage of the antenna system 10 before or during subsequent imaging may therefore be established.

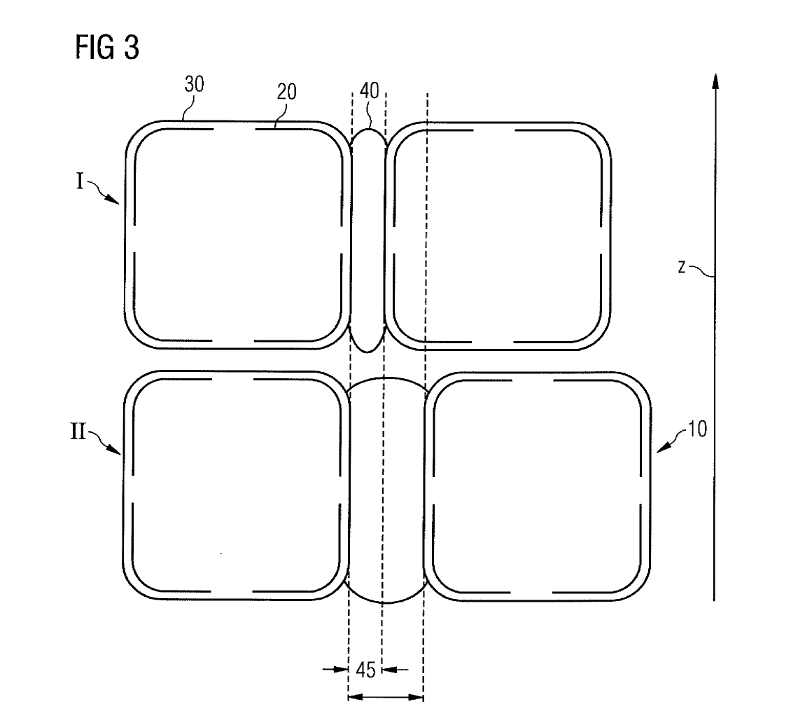

FIG. 3 shows a development of the antenna system 10 of FIG. 2 in detail. The antenna system 10, in contrast to the exemplary embodiment of FIG. 2, includes support films as the support elements 30.

The thickness of the connecting film, determined perpendicular to the flat side of the support film, may attain the thickness of the support film, so the antenna system 10 overall forms an approximately smooth, cohesive surface, so the antenna system 10 is easy to handle. The resulting flexibility and expandability is described in more detail below.

As shown in FIG. 3, a change in the length extent of the chain of receiving coils occurs over the expansion of the connecting elements 40. The spacing 45 between individual antenna elements 20 may vary between a minimum spacing in the initial position I and a maximum spacing in a maximum position II.

A minimum decoupling of the antenna elements 20 from each other is provided by a defined spacing 45 or air gap or gap between adjacent antenna elements 20 in the initial position I. With such “gap design” of local coils 10, the spacing in the initial position I is, for example, at least 20% of a mean diameter of the respective conductor loop of the antenna element 20. For antenna elements 20 with a substantially rectangular conductor loop (e.g., an approximately rectangular conductor loop with chamfered corners), the mean diameter Rₘ may be determined by

$${R}_{m} = D \cdot \frac{1}{\sqrt{5}} \cdot \frac{1}{\sqrt{( {n}^{2} + 1 ) \cdot 0 , 7826}} ,$$

where D is the diagonal of the rectangle, and n is the ratio of length to width of the rectangle.

The expandability of the connecting elements 30 may be limited, so, for example, a maximum spacing of 50% of the mean coil diameter results. A minimum covering of the object to be examined by antenna elements 20 is therefore provided.

The configuration of the receiving coil arrays with a spacing between the individual receiving coils as a “gap design” provides advantages. Gap design coils are limited in the “penetration depth” owing to the small “loop size” that is due to the spacing between adjacent antenna elements 20. The penetration depth is a measure for the effective range of the antenna element 20 that also determines the signal-to-noise ratio. The relatively “small” conductor loops of the antenna elements 20 of the gap design enable good adjustment of the local coil 10 to the surface shape of the object to be examined, however, so the filling factor may be drastically increased. The drawback of a lower penetration depth, for example, compared with “overlap coils” (illustrated later) may be compensated by the increase in the filling factor, and the signal-to-noise ratio reduced (e.g., lost) by the gap design may turn out to be equal again or, depending on the type of coil, even increased. In a synergetic manner, an advantage of the construction of the antenna system as a gap design results therefore.

This advantage is clarified below by a specific example. The antenna system shown in FIG. 2 may be secured by way of example as a local receiving coil array so as to surround a knee (e.g., form a “knee coil”). With optimum setting of the filling factor, which in the case of the knee coil is provided by the ratio of coil cross-sectional area to knee cross-sectional area, a margin of change in the signal-to-noise ratio up to a factor of 1.5 in the case of a knee diameter of 10-16 cm may be achieved, for example.

An optimum mean coil diameter R may be determined in this case for a desired penetration depth z by way of the formula (according to a dissertation by Arne Reykowski “Theory and Design of Synthesis Array Coils for Magnetic Resonance Imaging” submitted December 1996, Texas A&M University.)

Therefore, for a coil diameter of 4.4 cm (e.g., optimum for an approximately 10 cm thick knee in the transversal plane), for example, a filling factor of about 2.2 results. The signal-to-noise ratio is proportional to the root from the filling factor, so on the basis of the filling factor, a variation bandwidth, and therewith a potential improvement in the signal-to-noise ratio of 1.5 with respect to a signal-to-noise ratio provided by further factors of the coil, result purely by way of calculation.

Further advantages of a gap design antenna system result from an improved usefulness of the antenna system for parallel imaging methods.

In the case of the example of the described knee coil, six successive, independent antenna elements 20 arranged along the circumferential line of the knee are provided in the case of an optimum receiving coil diameter, calculated according to the above formula, of 4.4 cm and a spacing of the receiving coils of about 25% of the coil diameter for the above-described knee diameter. Owing to the good decoupling of the independent antenna elements 20 of the gap design, parallel magnetic resonance data acquisition may occur via the six independent antenna elements 20. The parallel data acquisition is described by the “PAT factor” (e.g., “PAT”), which may, for example, reach six.

An intrinsic property of local coils is that the coil profile is not constantly mapped in space in terms of amount and phase of a magnetic resonance signal. This is described by the “geometry factor” (e.g., g factor).

Gap design antenna systems are distinguished by low geometry factors and are therefore predestined for methods of parallel imaging. A signal-to-noise ratio (SNRp) that is reduced compared with the signal-to-noise ratio of sequential image acquisition (SNRs) in the case of parallel image acquisition may be described by formula

$$S N R p = \frac{S N R s}{g \cdot \sqrt{PAT}}$$

The possibility of accelerating parallel image data acquisition (e.g., increase in the PAT factor) with an acceptable signal-to-noise ratio (SNRp) is provided.

The “artifact power” behavior may also be improved with an antenna system according to the “gap design.” The transmitting or receiving profiles of adjacent antenna elements are separated, and ambiguous artifacts due to folds may be easily avoided.

With the aid of the described construction of the receiving coil array as a combination of support elements and connecting elements, which may result in a gap design, a series of unexpected advantages is therefore provided.

As already illustrated, the connecting films in the exemplary embodiment are constructed so as to be expandable, and the support elements 30 constructed as thin support films are constructed so as to be non-expandable, but likewise pliable. This provides that the surface dimension of the support films does not change in the case of bending, so the peripheral dimensions (e.g., of the external perimeter of the rectangular shape) are unchanged and constant in contrast to connecting films. Therefore, fixed transmitting or receiving properties of the respective antenna elements 20 allocated to the support films may be attained even with pliable antenna elements 20. Reliable operation with an optimum signal-to-noise ratio is therefore possible. For example, the antenna elements 20 virtually do not change loop size in the case of bending of their respective support element 30 (e.g., the loop size is substantially constant).

In the exemplary embodiment of FIG. 3, the materials of the support films and the connecting films differ, so expandability of the connecting film may be an intrinsic material property of the connecting film that does not include the support film, for example. The connecting film may have a thickness of between 0.1 mm and 0.2 mm for this purpose. In this case, a corresponding support film may have a thickness between 0.1 mm and 0.2 mm. The support film may also be connected in a planar manner to a foam material that encloses both the antenna element 20 and the support element 30.

Additionally or alternatively, the support films and the connecting films may be formed from identical materials. The support films may be provided with limiting elements, so expansion is prevented. For example, the limiting elements may be an encircling reinforcing ring made of a fiber providing tensile strength or the like, which is connected, for example, to the support film. The support elements 30 and the connecting elements 40 may also be constructed in one piece from a film in this respect, with the support elements 30 being separated or distinguished from the connecting elements 40 only by the limiting elements.

Beyond the diagram of FIG. 3, a plurality of rows of antenna elements 20 may be arranged perpendicular to the direction of expansion (e.g., in a z direction in FIG. 3), which, for example, is the direction of the basic magnetic field B₀ of the tomograph. These rows may include antenna elements 20 that, perpendicular to the direction of expansion, are arranged in each case so as to overlap antenna elements 20 of the adjacent rows. The overlapping in the z direction may be strictly predefined. With advantageous expandability of the antenna system, the filling factor may be improved further by a dense arrangement of the antenna elements in the z direction.

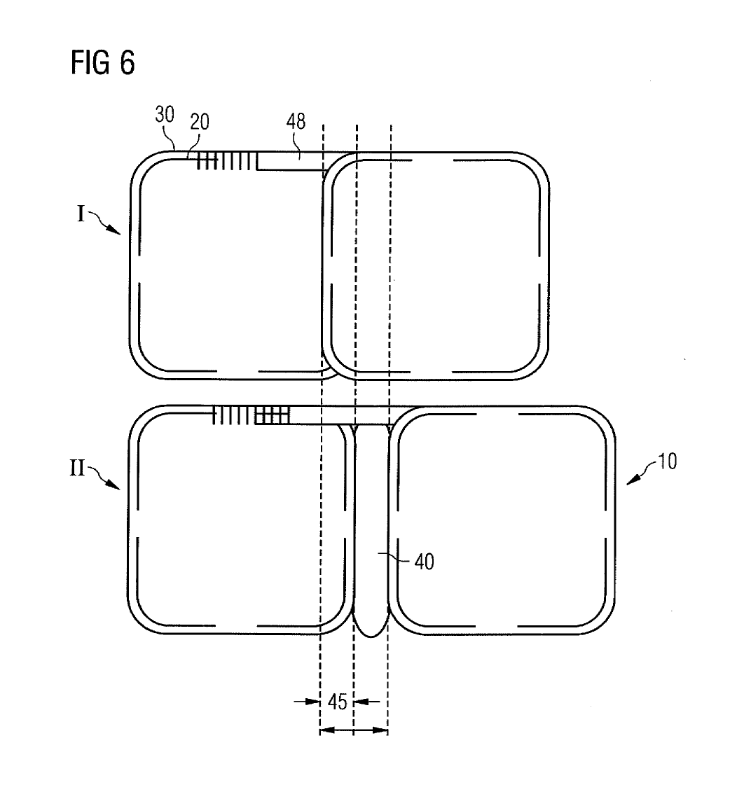

An alternative embodiment with variable overlapping is described in more detail below with, for example, the aid of FIG. 6.

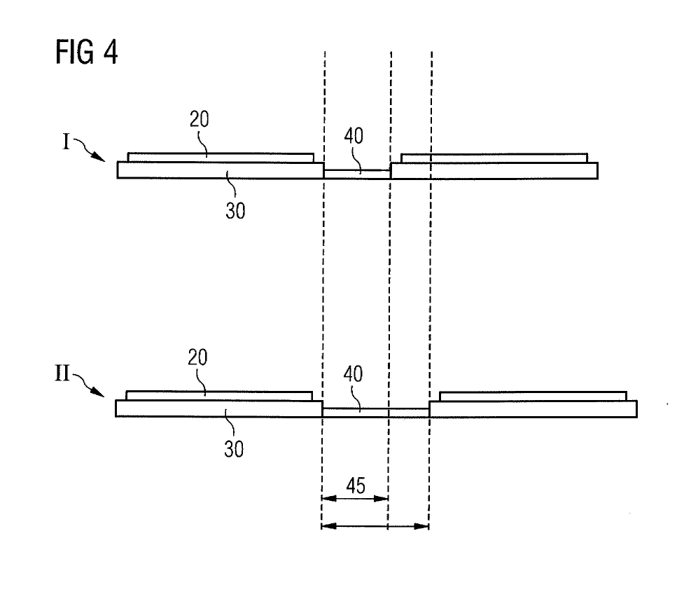

FIG. 4 clarifies the expansion of the local coil 10 in FIG. 3 in a cross-section. On a side facing the object to be examined (e.g., the lower side in FIG. 4), the antenna system 10 forms an approximately continuous surface in both initial position I and maximum position II. For this purpose, the connecting films are arranged flush with a side of the support elements 30 facing the object to be examined. This arrangement is retained with expansion of the connecting film from initial position I into maximum position II.

In contrast to the exemplary embodiment in FIG. 4, which shows a plurality of connecting elements 40, the support elements 30 may also be arranged on a single expandable, continuous connecting element 40 that forms a continuous smooth surface facing the object to be examined. The support elements may, for example, be glued all over to the surface facing away from the object to be examined or be let into the continuous connecting element 40 (e.g., cast).

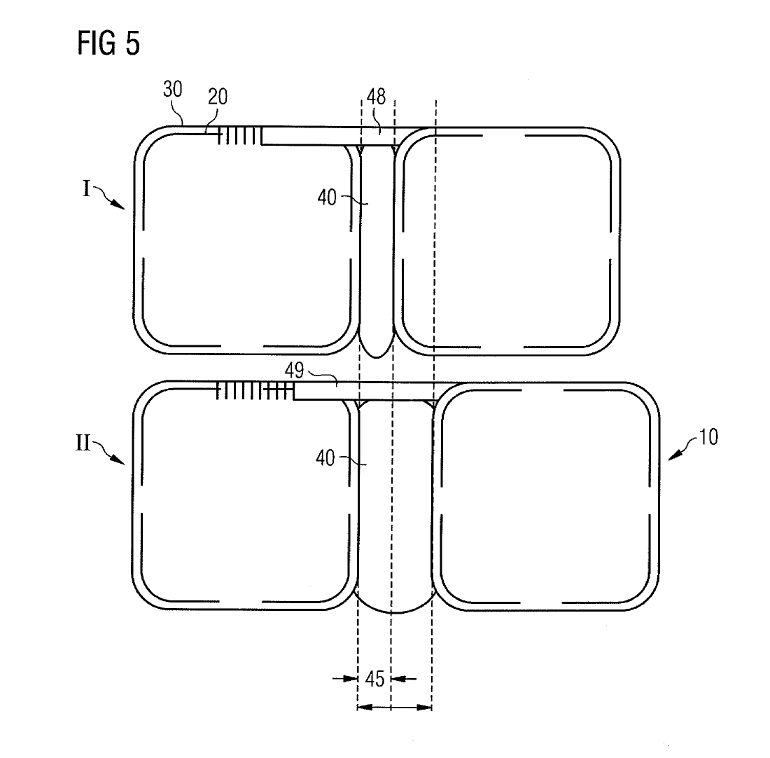

FIG. 5 shows one possibility for definitively setting the spacing between adjacent antenna elements 20. With the aid of a latching device 48 constructed as a ribbed hinge, in which latching noses 49 arranged on the support element 30 may engage, a series of positions are fixed for the expansion of the connecting element 40. The positions each correspond to a certain spacing between the antenna elements 20, which simplifies operation of the antenna system 10 without extensive adjustment measures.

For example, this may also be useful, as illustrated in FIG. 6, for antenna elements 20 that are arranged so as to overlap each other (e.g., in contrast to the above-described exemplary embodiments, implement an “overlap design” of the antenna system). In this case, with the aid of the positions of the spacing of the antenna elements 20 from each other, decoupling of these antenna elements 20 may be achieved by certain overlap positions of the conductor loops of adjacent antenna elements 20, so adjustment measures to the surface shape of an object to be examined result in a fixed signal quality. Simple operation of the antenna system is thus provided.

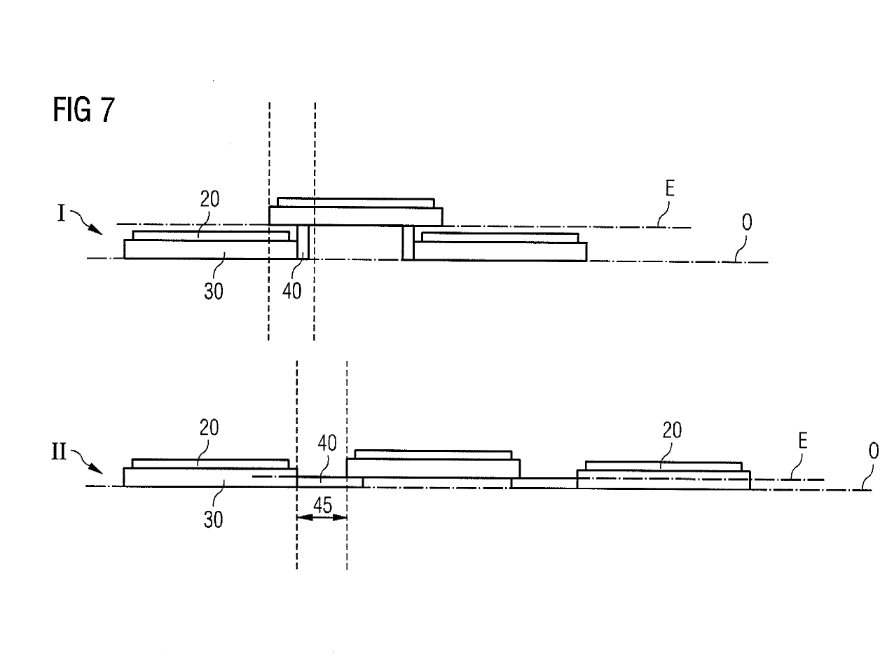

FIG. 7 shows a cross-section of a portion of such an antenna system 10 with an “overlap design.” In an initial position I, a plurality of support elements 30 are arranged in an “object plane” O that substantially follows the surface of an object to be examined. With these support elements 30 overlapping, further support elements 30 of the antenna system 10 are arranged offset with respect to the object plane. The overlap design also achieves a chain-like connection for adjacent antenna elements 20 respectively, although the connection is made in the initial position I via support elements 30 that are arranged adjacent in a plurality of planes. In the case of the overlap design, the connecting elements 30 extend in the initial position I from a first object plane O to a second plane E arranged slightly further away from the object to be examined. These planes O, E are arranged more closely over each other, so the support elements 30 with the connecting elements 40 located therebetween lie directly on each other. As shown in FIG. 7, the connecting elements 40 are not solely arranged on the limiting edges of the support elements 30. In the exemplary embodiment, this applies to the support elements 30 of the object plane O.

In a maximum position II, the connecting elements 40 may be expanded such that all support elements 30 or antenna elements 20 are arranged in the object plane O and have a spacing 45 from each other (e.g., correspond to a gap design). The antenna system 10 may therefore merge from an “overlap design” into a “gap design.”

For the case of at least partial use of the antenna system 10 as a receiving coil, decoupling of the relevant antenna elements 20 may also be decisively determined by the pre-amplifier decoupling. For this reason, a separate preamplifier for amplifying received magnetic resonance signals of the object to be examined is allocated to adjacent antenna elements 20, respectively, as described in more detail below. For example, this allocation enables advantageous decoupling of adjacent antenna elements 20, so, as described above, a transition that is free from complications may be achieved therewith between the design variants for antenna systems according to both the “overlap design” and the “gap design.”

Alternatively, a limit that limits an expansion of the connecting elements 40 such that the antenna elements 20 also overlap by a minimum amount in the maximum position II may also be provided, so a situation where the antenna system 10 may be changed from an “overlap design” to a “gap design” is ruled out.

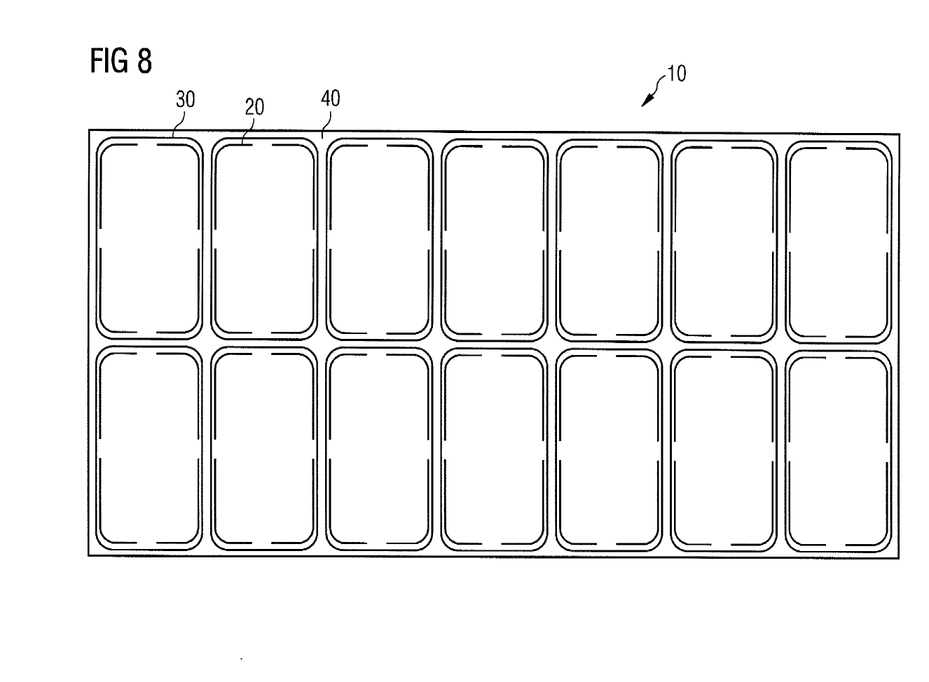

FIG. 8 shows a further exemplary embodiment. In this case, the antenna system 10 is constructed as a matrix-like arrangement of the antenna elements 20, already described in FIG. 3, which are arranged on support films. The connecting elements 30 are constructed as expandable connecting films. In contrast to the exemplary embodiment of FIG. 3, the connecting films are accordingly arranged between adjacent antenna elements 20 such that a cohesive, two-dimensional structure results in a plane that is, for example, rectangular and may be laid, for example, like a blanket over an object to be examined. In this connection, a “2D arrangement of the antenna elements” may also be provided. In this connection, the antenna elements 20 are arranged in a plane in a plurality of rows, in an initial position substantially spaced apart from each other in a uniform grid or with the same grid spacing. Connecting films are located between the rows of the antenna elements 20 or rows of support films, respectively, so a plurality of connecting films is arranged on a support film. The plurality of connecting films is arranged on the narrow side and on the long side of the support film, respectively, and enables an expansion in mutually orthogonal spatial directions.

Beyond the diagram of FIG. 8, the matrix-like arrangement may also be constructed such that a tunnel-, stocking-, glove- or tube-like antenna system 10 is formed. For example, a leg, an arm, a foot, a hand or an object to be examined with a similarly complex shape may be closely surrounded by the antenna system 10 without great effort. In the exemplary embodiment, the matrix-like 2D arrangement forms a cohesive network of antenna elements 20 that may encompass a three-dimensional body.

To improve fixing of handling of the antenna system 10, the antenna system 10 may include tensile or pressure elements (not shown). For this purpose, belts or the like, for example, may be secured to the side of the receiving coil array remote from the object to be examined, and these limit, tension and/or pretension expansion of the receiving coil array or the connecting films.

In one embodiment, the belts may run, similarly to the exemplary embodiment in FIG. 2, in the circumferential direction of the tunnel-like or the tube-like antenna system 10, which may closely and completely surround, for example, a leg or an arm. With the aid of the belts, an expansion, for example, of the connecting elements may be fixed by a plurality of support elements, so, for example, the local coil may rest closely in the hollow of a knee.

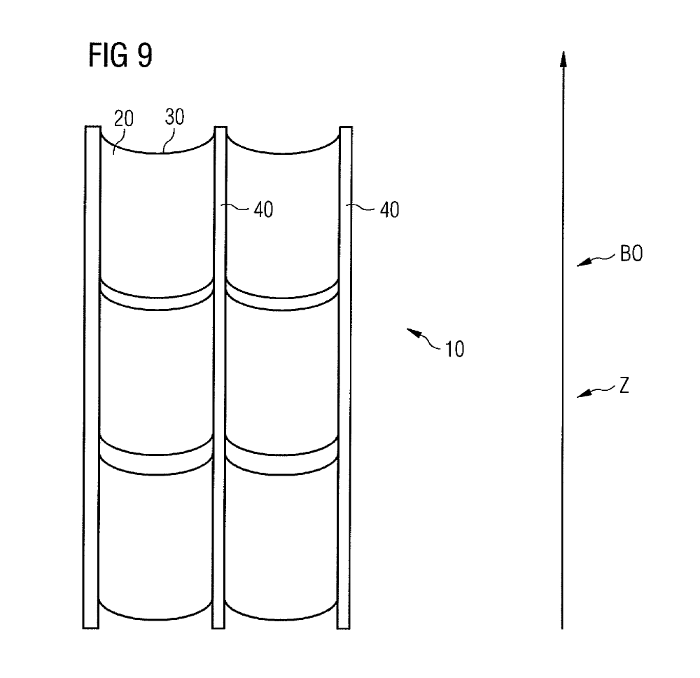

FIG. 9 shows a further embodiment of a support element 30 of FIG. 8. In the exemplary embodiment, the support films have a curved design (e.g., the support films have a curved surface section in an initial position). This surface section reproduces this shape of an object to be examined and thus enables optimum adjustment of the antenna system 10 to this surface shape. For example, this may be a section of a heel, a hand, a knee, a shoulder or a section of an object to be examined with a similarly complex shape.

The exemplary embodiment of FIG. 9 reproduces an arm or leg section through the shape of the support films. The local coil includes a plurality of support elements 30 that reproduce a section of a cylinder or a part of a circumferential surface of a cone, so all support elements 30 of the local coil, for example, have a curved surface section. The connecting elements 40 are configured in the form of connecting films as narrow webs between a plurality of rows of support films and are arranged parallel to a longitudinal axis of a fictive cylinder or cone that determines the form of the support films. In the exemplary embodiment, outstanding adjustment to approximately cylindrical or conical objects to be examined such as an arm or leg, for example, is therefore provided. Placed around a leg, for example, which is oriented parallel to a z direction, corresponding to a magnetic field direction of a basic magnetic field B₀ of the magnetic resonance imaging system, a plurality of antenna elements follow each other in the z direction in the antenna system 10. The antenna system 10 is therefore constructed with “z staggering.”

Irrespective of the specific construction of the individual connecting elements 40 and support elements 30 (e.g., irrespective of whether the elements are constructed so as to be pre-shaped or flat, side by side or overlapping), the extent of the antenna system 10 in the z direction (e.g., corresponding to the body axis of a patient to be examined) is, for example, at least 5% in an initial position, at least 10% or about 20% of the dimension in the z direction of a homogeneity volume of the basic magnetic field B₀ of the magnetic resonance imaging system. The dimension of the homogeneity volume in the z direction may not be exceeded by the antenna system. Effective parallel imaging is possible within the described limits, with the required flexibility simultaneously now being achieved. Similarly, the antenna system 10 is also constructed in such a way that, as a function of the positioning of the patient, even with maximum expansion in the x and/or y direction(s). These directions are orthogonal to each other and are each oriented orthogonally to the z direction. The antenna system may be arranged completely within the homogeneity volume of the tomograph, thus enabling maximum parallelism of the imaging.

The network of cohesive antenna elements 20 or support films 30 may, as shown in FIG. 8 or 9, be constructed as a regular arrangement of similar support elements 30 and antenna elements 20. This enables efficient operation of the antenna system 10 and minimizes the compensation expenditure for adjustment of the local coil 10 to an object to be examined. Cost advantages in the production of the local coil 10 may also be achieved.

The present embodiments are not limited to similar antenna elements 20 or support elements 30, however. Different types of antenna elements 20 and/or support elements 30 may also be linked to each other in such a matrix-like arrangement. The antenna elements 20 may have different dimensions, and a different basic shape is also conceivable.

For example, a combination of dedicated receiving antenna elements and dedicated transmitting antenna elements may also be present. In this case, this also includes the possibility of the dedicated receiving antenna elements each being constructed so as to be identical to each other and/or the dedicated transmitting antenna elements being to the same as each other.

The antenna system 10 therefore includes a plurality of groups of identical antenna elements 20 that may each be switched or operated separately.

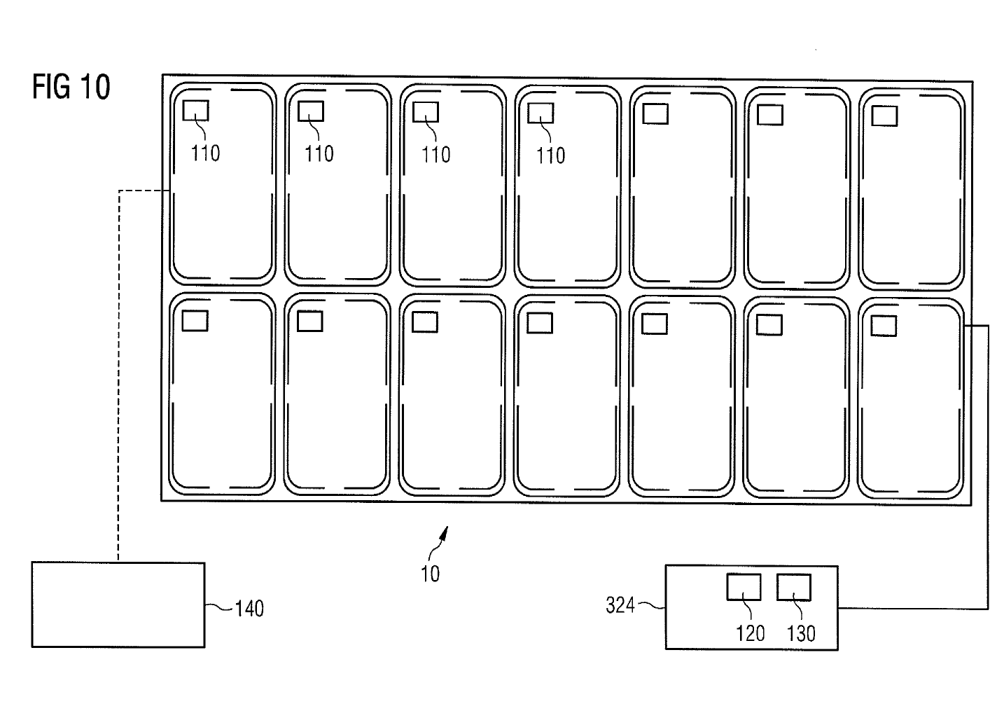

FIG. 10 shows a further exemplary embodiment of a local coil 10 that is again constructed as a receiving coil array and substantially matches the exemplary embodiment in FIG. 8.

In one embodiment, the receiving coil array is constructed for cableless operation (e.g., connecting lines to each of the antenna elements 20 of the arrays may be omitted, or the number of connecting lines may be reduced), so extensive measures for compensation of changed electrical lengths, caused by the expandable design of the antenna system 10 overall, are avoided.

As shown, a preamplifier 110, an analog-to-digital converter 120 and a modulator 130 are allocated to the antenna system 10, all of which may be connected to a transmitter for wireless information transmission to wirelessly transmit information to a receiving unit (cf., FIG. 1) of the magnetic resonance imaging system 1. All of these components may be part of the local coil transmitting device 324 or the local coil controller 322 described above in relation to FIG. 1. One preamplifier 110, respectively, is allocated to each antenna element 20 in the exemplary embodiment. The described components may also be present several times (e.g., allocated to one antenna element 20, respectively, or also for a plurality of groups of antenna elements 20).

The antenna elements 20 may be constructed for wireless control of tuning, so the resonance frequency of individual antenna elements 20 or of groups of antenna elements may be wirelessly activated (e.g., the natural frequency of the antenna element is tuned to the magnetic resonance frequency) or detuned. Passive detuning of the natural resonance frequency of the antenna elements 20 is provided (e.g., power for detuning pin diodes for tuning the natural resonance frequency is taken from an HF transmitting field of the magnetic resonance imaging system 1).

Additionally or alternatively, a transmitting coil array may also be constructed for cableless operation.

As shown by broken lines in FIG. 10, a group of antenna elements 20 or the entire antenna system 10 may be connected to a transmitting controller 140 constructed for wireless operation, and this controls the transmitting coils of the antenna system 10.

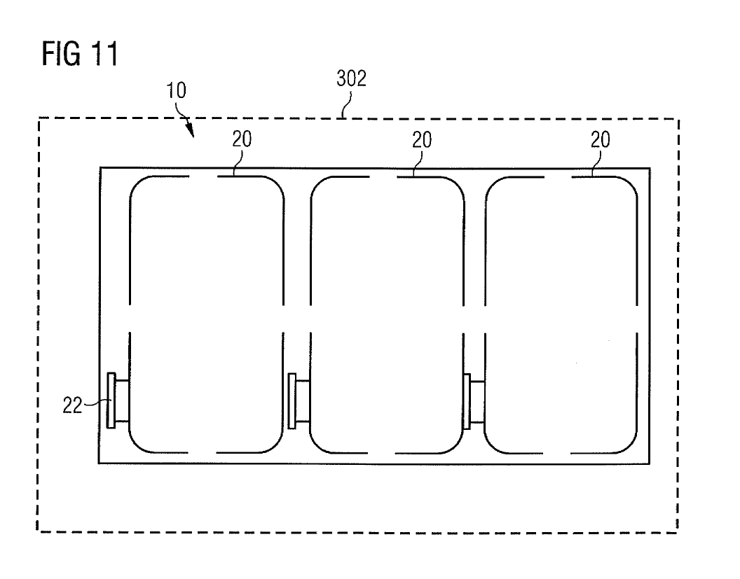

In this case, as is shown in the exemplary embodiment of FIG. 11, during transmitting mode, the transmitting coils may be inductively coupled to the transmitting antenna arrangement 302 described above in connection with FIG. 1 and shown only schematically. For this purpose, the transmitting coils include tuning elements 22 (e.g., tunable capacitors or tunable arrangements of capacitors), which enable defined coupling of the transmitting coils to the transmitting coil arrangement 302. With the aid of coupling, a fixed transmitting power may be transmitted to the local coil. The local coil radiates the fixed transmitting power with the natural frequency to the object to be examined. A separate supply line for transmitting operation to the individual antenna elements 20 may therefore be omitted.

The cableless or wireless operation of transmitting or receiving coils, for example, enables that electrical lengths may be kept constant during operation of the antenna system 10, so extensive tuning measures for operation of the antenna system may be omitted. This is advantageous, for example, since the partial expandability of the antenna system 10 seems to initially exclude constant electrical lengths.

The present embodiments effectively provide possibilities for significantly improving the adjustment of an antenna system to the surface shape of an object to be examined, as well as the signal-to-noise ratio of magnet resonance images (e.g., with parallel data acquisition), with the antenna system being easy to handle during operation.

The features of all exemplary embodiments or developments disclosed in figures may be used in any desired combination. The magnet resonance imaging systems or antenna systems described in detail above are only exemplary embodiments that may be modified in a wide variety of ways by the person skilled in the art without departing from the scope of the invention. Use of the indefinite article “a” or “an” does not prevent the relevant features from also being present several times.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An antenna system for a magnetic resonance imaging system, the antenna system comprising:
a plurality of antenna elements that are connected to support elements that each has a constant surface dimension,
wherein adjacent support elements of the support elements are connected by expandable connecting elements.

2. The antenna system as claimed in claim 1, wherein the support elements are constructed so as to be flexibly formable.

3. The antenna system as claimed in claim 2, wherein the support elements are pliable.

4. The antenna system as claimed in claim 1, wherein each of the expandable connecting elements comprises an expandable film.

5. The antenna system as claimed in claim 1, wherein an at least two-dimensionally cohesive network of antenna elements of the plurality of antenna elements is formed with the aid of a plurality of the expandable connecting elements.

6. The antenna system as claimed in claim 1, wherein a substantial portion of the support elements are flat support elements.

7. The antenna system as claimed in claim 6, wherein all of the support elements are flat support elements.

8. The antenna system as claimed in claim 1, wherein the support elements are constructed so as to substantially follow a surface shape of a section of an object to be examined.

9. The antenna system as claimed in claim 8, wherein the support elements have a surface curved in certain sections.

10. The antenna system as claimed in claim 1, further comprising a form fixing element that is constructed to change, fix, or change and fix an expansion of the antenna system.

11. The antenna system as claimed in claim 1, wherein antenna elements of the plurality of antenna elements are arranged at a minimum spacing from each other in an initial state of the antenna system.

12. The antenna system as claimed in claim 1, wherein antenna elements of the plurality of antenna elements are arranged so as to overlap each other in an initial state of the antenna system.

13. A magnetic resonance imaging system comprising:
an antenna system comprising:
a plurality of antenna elements that are connected to support elements that each has a constant surface dimension,
wherein adjacent support elements of the support elements are connected by expandable connecting elements.

14. The magnetic resonance imaging system as claimed in claim 13, further comprising antenna wiring, so the plurality of antenna elements are operatable cablelessly.

15. The magnetic resonance imaging system as claimed in claim 13, wherein the plurality of antenna elements is inductively coupled to a transmitting antenna arrangement of the magnetic resonance imaging system.

16. The magnetic resonance imaging system as claimed in claim 13, wherein the support elements are constructed so as to be flexibly formable.

17. The magnetic resonance imaging system as claimed in claim 16, wherein the support elements are pliable.

18. The magnetic resonance imaging system as claimed in claim 13, wherein each of the expandable connecting elements comprises an expandable film.

19. A method for the acquisition of magnetic resonance signals using a magnetic resonance imaging system, the method comprising:
emitting high frequency (HF) signals; and
receiving magnetic resonance signals of an object to be examined,
wherein an antenna system is used for transmitting the HF signals, receiving the magnetic resonance signals, or transmitting the HF signals and receiving the magnetic resonance signals, the antenna system comprising a plurality of antenna elements that are connected to support elements that each has a constant surface dimension, and
wherein adjacent support elements of the support elements are connected by expandable connecting elements.

20. The method as claimed in claim 19, wherein the antenna system is connected to a patient or test person before the patient or test person is positioned on a couch of the magnetic resonance imaging system.
